(19) United States
(12) Patent Application Publication
(10) Pub. No.: US 2023/0172466 A1
(43) Pub. Date: Jun. 8, 2023

(54) A Camera-Augmented FMCW Radar System for Cardiopulmonary System Monitoring

(71) Applicants: Autonomous Healthcare Inc., Hoboken, NJ (US); Stevens Institute of Technology, Hoboken, NJ (US)

(72) Inventors: Behnood Gholami, Menlo Park, CA (US); Negar Tavassolian, Menlo Park, CA (US); Arash Shokouhmand, Weehawken, NJ (US)

(73) Assignees: Autonomous Healthcare Inc., Hoboken, NJ (US); Stevens Institute of Technology, Hoboken, NJ (US)

(21) Appl. No.: 18/075,407

(22) Filed: Dec. 5, 2022

Related U.S. Application Data
(60) Provisional application No. 63/285,801, filed on Dec. 3, 2021.

(51) Int. Cl. A61B 5/024; A61B 5/11; A61B 5/0205; A61B 5/00; A61B 5/05; A61B 5/113

(52) U.S. Cl. CPC A61B 5/02427; A61B 5/1126; A61B 5/0205; A61B 5/7246; A61B 5/05; A61B 5/113

(57) ABSTRACT

The present disclosure describes a technology for contactless cardiopulmonary system monitoring, and more specifically, to the embodiment of exemplary system and method for detecting torso movements and estimating respiratory and heart rates. This invention leverages a depth sensor-equipped camera system to determine the human's anatomical landmarks. The estimated coordinates guide an FMCW radar to enhance the signal quality in the direction of the subject through a beam-steering technique, and extract the movements corresponding to the cardiopulmonary system. The movements are used to estimate respiratory and heart rates in a processing unit.

### CROSS-REFERENCE TO RELATED APPLICATIONS

The present application claims priority to U.S. Provisional Application No. 63/285801 filed Dec. 3, 2021, titled “A Camera-Augmented FMCW Radar System for Cardiopulmonary System Monitoring,” which is hereby incorporated-by-reference in its entirety.

### GOVERNMENT RIGHTS

The invention was made with government support under 80NSSC20C0117 by NASA. The government has certain rights in the invention.

### BACKGROUND

Current state-of-the-art vital sign monitoring systems widely used in patient monitoring such as inpatient wards include sensors directly attached to the patient's body to measure respiratory and heart rates. Contact-based approaches cause inconvenience for patients due to the obtrusive nature of the measurement procedure. In the past few years, radar-operated non-contact monitoring systems have been proposed. However, radar systems are highly prone to electromagnetic interferences and multi-path effects, which hinders the discrimination of humans from other objects, especially in closed environments such as homes, offices, inpatient wards, and any enclosed space such as a spacecraft during long duration space flight.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the disclosure provides a system to estimate heart rate and/or respiratory rate of a subject comprising: a) an optical camera and a depth camera configured to receive optical and depth data of the subject in the field of view; b) a camera processing unit communicatively coupled with the optical camera and the depth camera and a radar processing unit, wherein the camera processing unit extracts at least one anatomical landmark from the body of the subject based at least upon the optical data; c) a radar configured to send and receive a radar signal; and d) a radar processing unit communicatively coupled with the radar and the camera processing unit to identify a point of interest on the subject's torso and extract heart rate and respiratory rate based at least upon the movement of the point of interest. In some embodiments, the disclosed system further comprises a database module to record at least the estimated heart rate and/or respiratory rate of the subject.

In some embodiments, the disclosure provides a computer-implemented method for estimating heart rate and/or respiratory rate of a subject, the method comprising: a) receiving optical and depth data of the subject in the field of view; b) extracting at least one anatomical landmark from the body of the subject based at least upon the optical data; c) sending a radar signal; d) receiving a radar signal and using the radar signal to identify a point of interest on the subject's torso; and e) extracting heart rate and respiratory rate based at least upon the movement of the point of interest.

### BRIEF DESCRIPTION OF THE FIGURES

Further objects, features and advantages of the present disclosure will become apparent from the following detailed description taken in conjunction with the accompanying figures showing illustrative embodiments of the present disclosure, in which:

FIG. 1 is an exemplary diagram of the camera-radar system for vital sign monitoring in accordance with certain exemplary embodiments of the present disclosure;

FIG. 2A is a flowchart of a video processing framework for the estimation of the subject's coordinates in accordance with certain exemplary embodiments of the present disclosure; FIG. 2B is a flowchart of a radar signal processing framework for respiratory and heart rates estimation in accordance with certain exemplary embodiments of the present disclosure;

FIG. 3A shows an exemplary region of interest for a point on the torso in accordance with certain exemplary embodiments of the present disclosure; FIG. 3B shows an exemplary set of points detected for left shoulder, right shoulder, left hip and right hip in accordance with certain exemplary embodiments of the present disclosure; FIG. 3C shows an exemplary region of interest for the centroid of the region defined by left shoulder, right shoulder, left hip, and right hip in accordance with certain exemplary embodiments of the present disclosure; FIG. 3D shows an exemplary plurality of points of interests in accordance with certain exemplary embodiments of the present disclosure; FIG. 3E shows an exemplary region of interest for the smaller sub areas on the torso in accordance with certain exemplary embodiments of the present disclosure;

FIG. 4 is an exemplary schematic diagram of the radar antennas and their relative distances to the camera sensors in accordance with certain exemplary embodiments of the present disclosure;

FIG. 5A is a graph of representative de-ramped linear-frequency modulated (LFM) signal in accordance with certain exemplary embodiments of the present disclosure; FIG. 5B is a graph of representative range profile signal in accordance with certain exemplary embodiments of the present disclosure;

FIG. 6A is an exemplary range-time profile in accordance with certain exemplary embodiments of the present disclosure; FIG. 6B is an exemplary range-time profile after DC compensation in accordance with certain exemplary embodiments of the present disclosure;

FIG. 7A is an exemplary movement signal in accordance with certain exemplary embodiments of the present disclosure; FIG. 7B is the unwrapped exemplary movement illustrated in FIG. 7A in accordance with certain exemplary embodiments of the present disclosure;

FIG. 8A is an exemplary respiratory signal in accordance with certain exemplary embodiments of the present disclosure; FIG. 8B is an exemplary heartbeat signal in accordance with certain exemplary embodiments of the present disclosure;

FIG. 9A is the spectrum of an exemplary respiratory signal in accordance with certain exemplary embodiments of the present disclosure; FIG. 9B is the spectrum of an exemplary heartbeat signal in accordance with certain exemplary embodiments of the present disclosure; and FIG. 9C is the spectrum of an exemplary heartbeat signal after the cancellation of the respiratory harmonics in accordance with certain exemplary embodiments of the present disclosure.

Throughout the drawings, the same reference numeral, and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the embodiments illustrated in the figures.

### DETAILED DESCRIPTION

Any algorithm described herein can be embodied in software or set of computer-executable instructions capable of being run on a computing device or devices. The computing device or devices can include one or more processor (CPU) and a computer memory. The computer memory can be or include a non-transitory computer storage media such as RAM which stores the set of computer-executable (also known herein as computer readable) instructions (software) for instructing the processor(s) to carry out any of the algorithms, methods, or routines described in this disclosure. As used in the context of this disclosure, a non-transitory computer-readable medium (or media) can include any kind of computer memory, including magnetic storage media, optical storage media, nonvolatile memory storage media, and volatile memory. Non-limiting examples of non-transitory computer-readable storage media include floppy disks, magnetic tape, conventional hard disks, CD-ROM, DVD-ROM, BLU-RAY, Flash ROM, memory cards, optical drives, solid state drives, flash drives, erasable programmable read only memory (EPROM), electrically erasable programmable read-only memory (EEPROM), non-volatile ROM, and RAM. The computer-readable instructions can be programmed in any suitable programming language, including JavaScript, C, C#, C++, Java, Python, Perl, Ruby, Swift, Visual Basic, and Objective C. Embodiments of the invention also include a non-transitory computer readable storage medium having any of the computer-executable instructions described herein.

A skilled artisan will further appreciate, in light of this disclosure, how the invention can be implemented, in addition to software and hardware, using one or more firmware. As such, embodiments of the invention can be implemented in a system which includes any combination of software, hardware, or firmware. In the context of this specification, the term “firmware” can include any software programmed onto the computing device, such as a device's nonvolatile memory. Thus, systems of the invention can also include, alternatively or in addition to the computer-executable instructions, various firmware modules configured to perform the algorithms of the invention.

According to embodiments, the computing device or devices can include a mainframe computer, web server, database server, desktop computer, laptop, tablet, netbook, notebook, personal digital assistant (PDA), gaming console, e-reader, smartphone, or smartwatch, which may include features such as a processor, memory, hard drive, graphics processing unit (GPU), and input/output devices such as display, keyboard, and mouse or trackpad (depending on the device). Embodiments can also provide a graphical user interface made available on one or more client computers. The graphical user interface can allow a user on a client computer remote access to the method or algorithm.

Additional embodiments of the invention can include a networked computer system for carrying out one or more methods of the invention. The computer system can include one or more computing devices which can include a processor for executing computer-executable instructions, one or more databases, a user interface, and a set of instructions (e.g., software) for carrying out one or more methods of the invention. According to other embodiments, the computing device or devices can be connected to a network through any suitable network protocol such as IP, TCP/IP, UDP, or ICMP, such as in a client-server configuration and one or more database servers. The network can use any suitable network protocol and can be any suitable wired or wireless network including any local area network, wide area network, Internet network, telecommunications network, Wi-Fi enabled network, or Bluetooth enabled network.

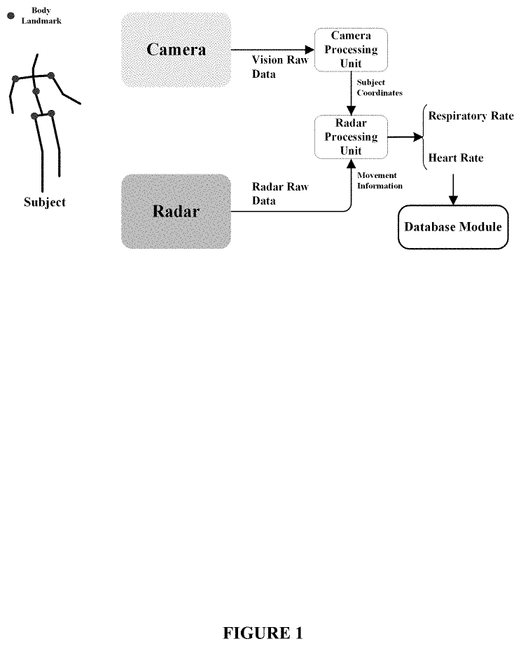

FIG. 1 shows the camera-radar system for cardiopulmonary state monitoring of one or more subjects. A frequency-modulated continuous-wave (FMCW) radar is used to sense the environment and extract movement information. Furthermore, a camera that consists of red-green-blue (RGB) and depth sensors is employed to estimate the subject's body pose and body landmarks. A camera processing unit processes video and depth data to estimate one or more subjects body pose and body landmarks and provides the subject or subjects coordinates to the radar processing unit. A radar processing unit processes radar signals, to extract chest wall movements to estimate respiratory and heart rates. The radar processing unit spatially filters the radar raw data in the direction of subject's torso, the coordinates of which are provided by the camera processing unit, to enhance and extract the movement information. In some embodiments, the estimated heart rate and/or respiratory rate values and timestamps associated with the time of those estimations are recorded in a database module that is in communication with the radar processing unit and camera processing unit. In some embodiments the database module is in communication with an external software through an application programming interface to report or transmit the estimated values of heart rate, respiratory rate, and timestamps.

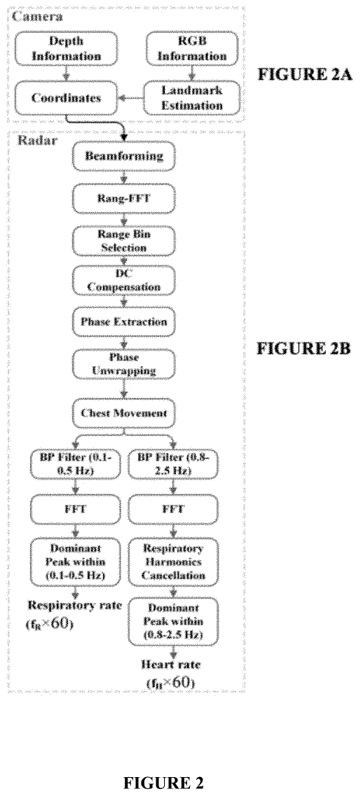

The camera processing unit analyzes RGB (or other grayscale or color images) and depth videos (FIG. 2A). These include a set of optical image frames from an optical (e.g., RGB) camera and a set of depth frames from a depth camera. Depth data may be measured with different techniques such as time of flight, structured light, and stereo vision among other methods. In some embodiments, the depth sensor employed in this framework operates based on the time-of-flight (ToF) framework, which estimates the z values by the time it takes the light to be emitted from the projector towards the environment and received by the depth sensor. In some embodiments, structured light is used for measuring depth. In some embodiments, computer vision techniques related to stereo vision is used to measure depth.

The optical and depth videos are used to identify one or more subjects in the field of view and estimate the body pose or human pose (i.e., identifying and classifying the location of the joints in the human body) and identify anatomical landmarks visible to the camera. We can use Cartesian coordinates (x,y,z) to locate the exact location of a point of interest (e.g., an anatomical landmark or body joint) in the physical three-dimensional space. In some embodiments x and y coordinates of the various anatomical landmarks are detected using the optical data. In some embodiments x and y and z coordinates of the various anatomical landmarks are detected using the depth data. In some embodiments, the depth sensor is used to find the 3^{rd} physical coordinate (z) in space, where (x,y) coordinates are identified using optical or depth data.

In some embodiments, a machine learning classifier is used to identify the coordinates of the subject based on optical or depth frames. An example classifier to estimate human pose includes neural networks. In some embodiments, pose estimation frameworks described in Cao, Zhe, et al. “Realtime multi-person 2d pose estimation using part affinity fields.” Proceedings of the IEEE Conference on Computer Vision and Pattern Recognition. 2017 and Xiao, Bin, Haiping Wu, and Yichen Wei. “Simple baselines for human pose estimation and tracking.” Proceedings of the European Conference on Computer Vision (ECCV). 2018 can be used.

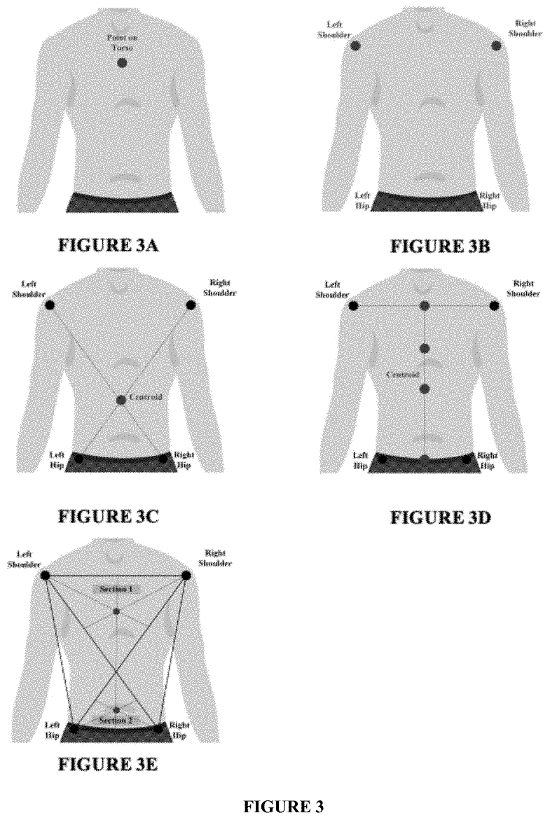

The landmarks identified by the pose estimation algorithm are 2-dimensional. As described above, the 3^{rd} dimension is identified using the one-to-one mapping of (x,y) to (x,y,z) coordinates intrinsic to the optical and depth cameras. The detected coordinates of the landmarks are used to identify one or more points of interest. In some embodiments, the point of interest is located on the torso of the subject (FIG. 3A). In some embodiments, left shoulder, right shoulder, left hip and right hip is detected by the camera processing unit (FIG. 3B). In some embodiments, the centroid of the region defined by left shoulder, right shoulder, left hip, and right hip is used as a point of interest (FIG. 3C). In some embodiments, a plurality of points of interest are identified (FIG. 3D). In some embodiments, the area defined by left shoulder, right shoulder, left hip, and right hip is divided into smaller sub areas and the centroid or any other point inside those areas are selected as a point of interest (FIG. 3E).

The coordinates of the detected point of interest are used by the radar processing unit to extract movement information from signal received by radar. FIG. 2B shows the flowchart for the radar processing unit. The FMCW radar is triggered to generate a linear frequency modulated (LFM) signal which is transmitted towards the environment. The LFM signal is configured to linearly sweep a certain frequency range, and is capable of modulating the movement information in the phase of the signal.

The transmitted LFM signal is characterized by a chirp rate and an initial frequency which are denoted by β and α respectively, as mentioned below:

x_{T}(t)=A_{T}exp(jπβt²+j2παt), (1)

where A_{T} and x_{T}(t) represent the amplitude and the transmitted chirp, respectively. Assuming a noise-less channel, x_{T}(t) is reflected towards the receiving antenna with a round-trip time of T, and de-ramped (de-modulated) by mixing the signal with a replica of the transmitted signal at the receiver. The de-ramped signal can be approximated as follows:

x*_{T}(t)x_{R}(t,T)≈A_{T}A_{R} exp(−j2πβTt−j2παT), (2)

where x*_{T}(t), x_{R}(t,T), and A_{R} represent the complex-conjugate of the transmitted signal, the received signal which is delayed by T, and the received power, respectively. The de-ramped signal represents a single-tone waveform with a frequency of f₀=βT and an initial phase of ϕ=αT. Hence, the Fourier transform of the mixed signal is called the range profile, which is equal to a Dirac delta function centered at f₀ in the frequency domain with an initial phase ϕ, as mentioned below:

[symbol]

{x*_{T}(t)x_{R}(t, T)}=A_{T}A_{R} δ(f+βT)exp(−j2παT)=A_{T}A_{R} δ(f+f₀)exp(−j2πϕ), (3)

where δ(.) and [symbol] {.} show the Dirac delta function and the Fourier transform, respectively. Since small movements around the fixed point corresponding to the subject's distance are induced by cardiopulmonary vibrations, the round-trip time can be expressed as

$$T = \frac{2 R + Δ r ( t )}{c} ,$$

with R and c representing the fixed distance from the subject's chest to the radar and the light speed, respectively. Furthermore, Δr(t) implies the small movements induced by the cardiopulmonary system movements. Substituting the expanded term of round-trip time into the estimated f₀ and ϕ results in

$${f}_{0} = β ( \frac{2 R + Δ r ( t )}{c} ) \approx \frac{2 β R}{c} and$$

$$ϕ = α ( \frac{2 R + Δ r ( t )}{c} ) = \frac{2 R α}{c} + {ϕ}_{0} ,$$

respectively. Δr(t) can be neglected in f₀ as it holds a significantly smaller value than R, and therefore does not shift the corresponding frequency range bin. Conversely, small chest movements are represented by ϕ₀=αΔr(t)/C in ϕ, which is not negligible as it appears in the phase which represents the movement. As a result, the movement can be calculated by performing Fourier transform on the de-ramped signal, which results in a dominant peak at the range bin associated with the distance of the subject. The phase variation of this range bin along the successive beat signals (also known as the slow-time axis) amounts to the temporal evolution of the chest movement.

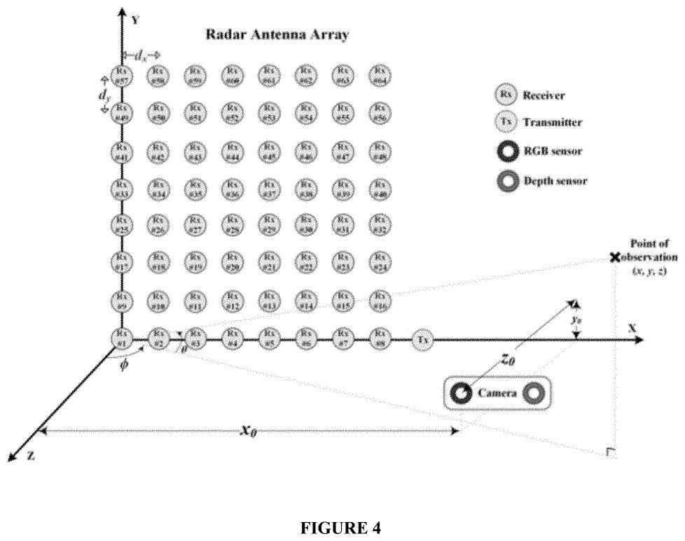

FIG. 4 shows an exemplary schematic diagram of the radar antenna array and its relative distance to the camera sensors. The radar consists of a single transmit (Tx) antenna and 64 receiving (Rx) antennas, the arrangement of which is presented in FIG. 4. The 64 Rx antennas are equidistantly spaced apart by dₓ and d_{y} along the X and Y axes, respectively. This exemplary structure allows for discriminating the movements associated with subjects located at a variety of azimuth (ϕ) and elevation (θ) angles with respect to the radar. In some embodiments, this architecture is implemented by defining virtual receiving channels through multiple-input multiple-output (MIMO) structures. In some embodiments, the number of Rx (or virtual receiving channels) can be different than 64. Any point of observation in a 3-D space is defined by a triplet (r, ϕ, θ) with r, ϕ, and θ representing the distance, the azimuth angle, and the elevation angle associated with a subject in FIG. 4, respectively.

As mentioned earlier, the coordinates of one or more points of interest on the subject are provided by the camera processing unit to the radar processing unit. However, the center point of the camera and its corresponding axes are not aligned with those of the radar. In this invention, the radar and the camera sensors are assumed to be consolidated into an enclosure and spaced apart by a predefined distance. This setup allows for triangulating the coordinates acquired by the camera into the radar coordinate system. Assuming (x, y, z) are the coordinates of a point of interest on the subject provided by the camera processing unit, the corresponding coordinates from the radar viewpoint will be (\hat{x}, ŷ, \hat{z})=(x−x₀, y−y₀, z−z₀), where x₀, y₀, and z₀ denote the difference between the radar and camera center points that should be offset as shown in FIG. 4. The antenna arrangement in this figure describes an 8×8 uniform rectangular array (URA). If the phase reference point is located at the origin (x, y)=(0,0), then the phase of the wave received at the element with coordinates (mdₓ,nd_{y}) can be determined as follows:

$${ϑ}_{m , n} ( ϕ , θ ) = \frac{2 π}{λ} ( m {d}_{x} cos θ sin ϕ + n {d}_{y} sin θ ) , \qquad ( 4 )$$

where ϕ=arctan(\hat{x}/\hat{z}) and θ =arctan(ŷ/ [symbol] ), and λ=c/f_{c} denotes the wavelength corresponding to the carrier frequency (f_{c}) of the transmitted signal. Therefore, the steering vector matrix is given by v(ϕ, θ)=[e^{-j∂}^{_{m,n}}]^{T}, with v*representing the direction of arrival (DoA) for antenna elements in a URA, which is associated with relative delays in receiving a reflected signal.

As shown in FIG. 2B, the radar signal processing algorithm begins with beamforming. As mentioned earlier, radar signals are contaminated by noise, interference, and multi-path components while modulating the movement information in the phase. To nullify the noise and interference, a beam-steering technique is applied on all 64 received signals. Assuming the 64 beat signals are contaminated by complex additive colored Gaussian noise N_{64×L} as follows:

Y_{(64×L)}=v_{(64×1)}a_{(1×L)}+N_{(64×L)}, (5)

where a and v represent the radar signal and the corresponding directions of arrival, respectively, with the covariance matrix of noise being S_{N}= [symbol] {NN^{H}} and {.}^{H} denoting the Hermitian operator. In some embodiments, the minimum-variance distortion-less response (MVDR) beam-steering technique described in Capon, Jack. “High-resolution frequency-wavenumber spectrum analysis.” Proceedings of the IEEE 57.8 (1969): 1408-1418 can be used as an exemplary method to coherently combine all 64 signals and enhance the signal quality in the subject's direction provided by the camera. The beam-steering vector can be calculated by

$$w = \frac{{S}_{N}^{- 1} v}{{v}^{H} {S}_{N}^{- 1} v} ,$$

where S_{N}⁻¹ denotes the inverse covariance matrix of noise. The weights are multiplied by their corresponding antennas beat signals and a single de-ramped signal is produced. Applying fast Fourier transform (FFT) on the de-ramped signal leads to the range profile signal.

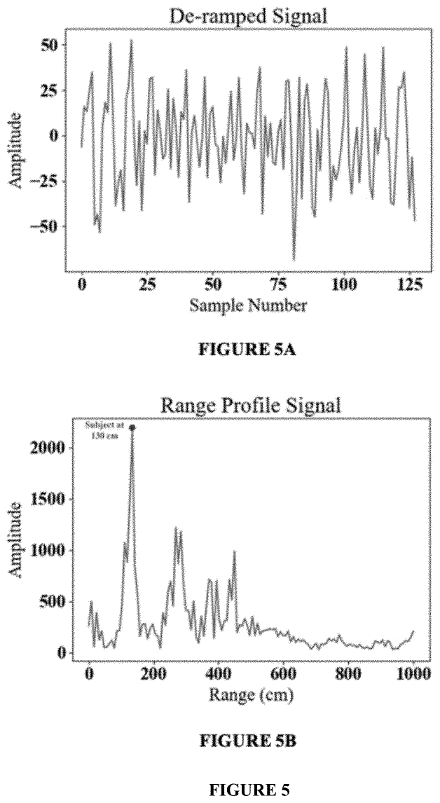

FIG. 5A and 5B depict an exemplary de-ramped signal and its corresponding range profile signal, respectively, for a subject located at a distance of 130 cm from an FMCW radar. The de-ramped signal in FIG. 5A follows a sinusoidal pattern as demonstrated in (2). The exemplary peak shown in FIG. 5B corresponds to the location of the subject in the range profile signal. The discussion above used 64 signals for demonstration purposes, however, the same framework can be used for any other antenna arrangement and resulting number of signals.

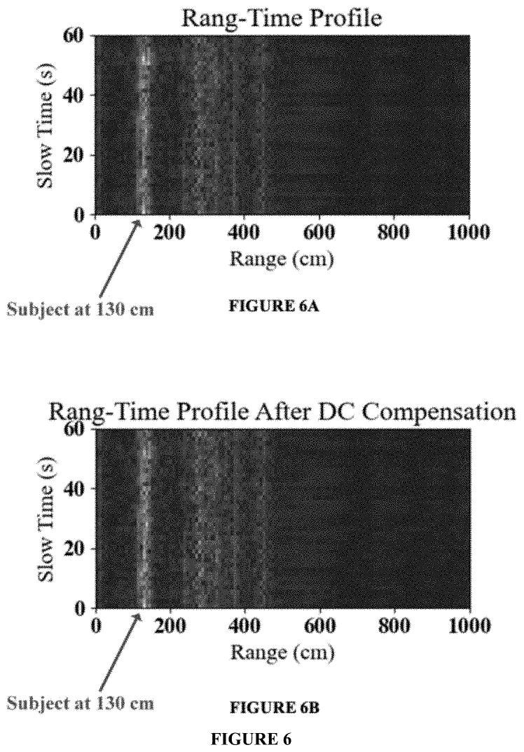

To obtain the time evolution of the chest movement, range profiles corresponding to successive de-ramped signals are arranged into a matrix with each row and column representing a range profile signal and a range bin, respectively. The constructed matrix is called a range-time profile consisting of slow time and range axes, as shown in FIG. 6A. Each range bin corresponding to a column in the range-time matrix represents the movement information at a certain distance from the radar. The exemplary range bin spiking in FIG. 5B that corresponds to a subject at 130 cm from an FMCW radar holds higher energy than other range bins in the range-time profile depicted in FIG. 6A. It is to be noted that we do not have to investigate all range bins to detect the subject since an approximate estimation of the subject's distance is provided by the camera. Therefore, the computational complexity of the method decreases dramatically as a result of using coordinates of one or more points of interest identified using depth information by the camera processing unit.

The coordinates provided by the camera correspond to the uppermost layer surface of the subject, which may include clothing. This is due to the fact that the depth sensor uses light with very low penetration into surfaces. In the case where the subject is wearing clothing, tracking the movement of the uppermost layer rather than the chest wall itself would reduce the accuracy since clothing may attenuate chest wall movements making such subtle movements more difficult to detect from uppermost layer surface movements. To determine the exact point representing the chest wall movement in a 3-D space, a search algorithm based on either signal power or the degrees of periodicity is used. The signal power algorithm compares energy levels of the signals corresponding to the range bins for higher distances than (but in the vicinity of) the subject's distance estimated by the camera. The signal with the highest energy level is selected as the source of movement, an example of which is illustrated in FIG. 6A. As for range bin detection based on the periodicity level, a method based on singular values of the signal can be used to determine the degrees of periodicity.

Let's assume a single-tone periodic signal h[k], k=1, 2, 3, . . . , l, with a period duration of T and signal length of l. We can reshape the signal into an m×L matrix with each row representing a single cycle (L=T) as below:

$${H}_{( m \times L )} = [ \begin{matrix}h ( 1 ) & … & h ( L ) \\ ⋮ & ⋱ & ⋮ \\ h ( ( m - 1 ) L + 1 ) & … & h ( m L )\end{matrix} ] . \qquad ( 6 )$$

Since h[k] is a periodic signal, the rows of the reshaped matrix H are dependent vectors, leading to a matrix of rank one. By performing singular value decomposition (SVD), H is decomposed into three matrices U_{(m×m)}, Σ_{(m×L)}, and V_{V(L×L)} as H=UΣV*, where Σ represents a diagonal matrix including the singular values of H on the main diagonal as given below:

$$Σ = [ \begin{matrix}{μ}_{1} & 0 & 0 & & \\ 0 & {μ}_{2} & 0 & … & 0 \\ 0 & 0 & {μ}_{3} & & \\ & ⋮ & & ⋱ & ⋮ \\ & 0 & & … & 0\end{matrix} ] . \qquad ( 7 )$$

The number of non-zero elements in Σ equals the rank of H. Therefore, H consists of only one non-zero element, i.e., μ₁, if it is a periodic signal. In this case, other elements, i.e., μ₂,μ₃, μ₄, etc. converge to zero. As a result, the ratio of the largest to the second-largest singular values

$$( \frac{{μ}_{1}}{{μ}_{2}} )$$

converge to infinity. It should be noted that this phenomenon happens if and only if the length of the rows (L) in H equals the period duration of the signal, i.e., L=T. This formulation can be extended to quasi-periodic signals, such as the chest movements induced by the cardiopulmonary system activities. As for quasi-periodic signals, the ratio of the largest to the second-largest singular value is expected to be a large number given that the length of the rows is selected equal to the period of the signal. However, if the assumed period is different than the actual period of the signal, the rows of H will be independent, and thus, the ratio of the singular values will not be a large value. In this work, to locate the most representative point, the ratio μ₁/μ₂ is calculated for the range bins as selected for the method for signal power. For this purpose, the ratio is calculated in terms of the assumed periods for the respiratory (2-10 s) and the heartbeat (0.5-1.25 s) signals. The point resulting in the maximum ratio, i.e., revealing the highest degrees of periodicity, is selected as the desired point.

The phase of the column corresponding to the subject's range bin represents the chest movement. However, the corresponding range signal is often corrupted by direct current (DC) values of external interferences, i.e., ∈_{I} and ∈_{Q}, which are associated with in-phase (I) and quadrature (Q) signal components, respectively. These DC terms cause the movement signal to be distorted. Hence, a strategy for pre-empting phase distortion caused by DC is required. Given I(t)=A_{T}A_{R} cos(−j2πϕ)+∈₁ and Q(t)=A_{T}A_{R} sin(−j2πϕ)+∈_{Q}, the following term is achieved by reformulating the relationship between I(t) and Q(t) :

$${\vert \frac{I ( t ) - {ϵ}_{I}}{{A}_{T} {A}_{R}} \vert}^{2} + {\vert \frac{Q ( t ) - {ϵ}_{Q}}{{A}_{T} {A}_{R}} \vert}^{2} = 1 , \qquad ( 8 )$$

which defines a circular constellation centered at (∈_{I}, ∈_{Q}). To correct the constellation, the DC terms should be determined. To this end, an optimization problem is defined by rearranging (8):

min{|{square root over ((l)(t)−∈_{I})²+(Q(t)−∈_{Q})²)}−A_{T}A_{R}|}, (9)

which is minimized through a gradient descent in terms of ∈_{I}, ∈_{Q}, and A_{T}A_{R}. As such, DC terms are offset by shifting and scaling the in-phase and quadrature components with respect to the optimum values of ∈_{I} and ∈_{Q}, and A_{T}A_{R}, respectively. FIG. 6B illustrates the range-time profile after DC compensation of the exemplary range-time profile in FIG. 6A.

After DC compensation, the phase of the signal is calculated by arctan

$$\frac{Q ( t )}{I ( t )} ,$$

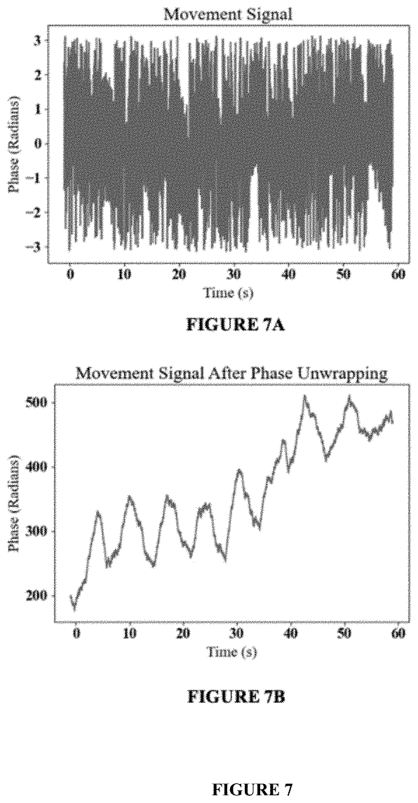

which provides the movement information. An exemplary movement signal is shown in FIG. 7A. The chest movement induced by the cardiopulmonary system falls within the range of 0.05-12 mm, and thus could potentially exceed the phase range ((−π, +π)) obtained by the atan 2{. } function. The excess movement value causes distortion as observed in FIG. 7A. To address this issue, the movement signal is unwrapped by comparing every two consecutive phase samples and checking their respective difference. If the difference exceeds the normal phase range ((−π, +π)), the phase of the latter sample is compensated by either adding/subtracting 2π. The movement signal in FIG. 7A after unwrapping is shown in FIG. 7B.

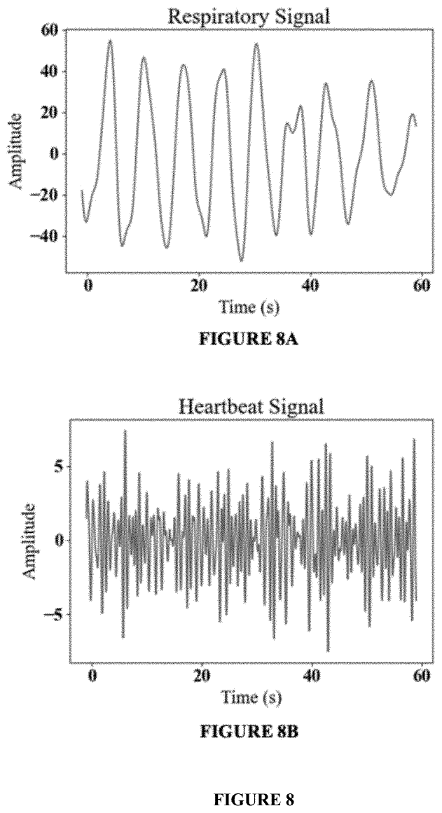

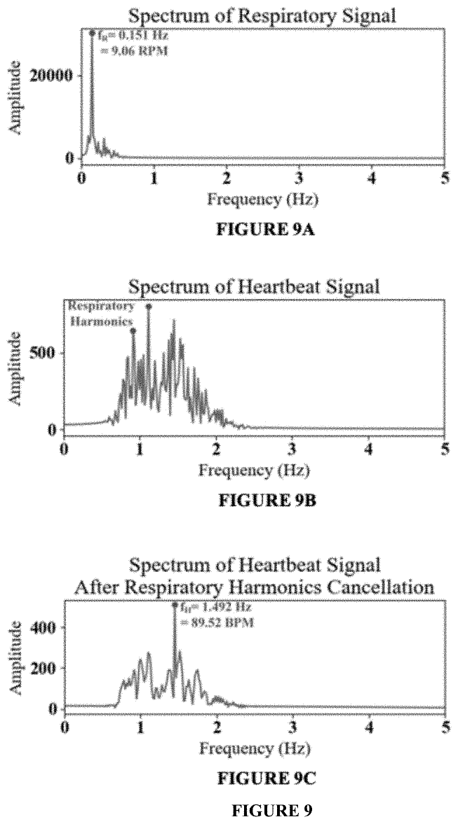

To obtain the respiratory and heartbeat signals, the unwrapped phase is band-pass (BP) filtered within the ranges of 0.1-0.5 Hz and 0.8-2.5 Hz, respectively. The examples of the extracted respiratory and heartbeat signals are shown in FIGS. 8A and 8B, respectively, where each peak represents a cycle. The energy of a respiratory signal is relatively higher compared to its corresponding heartbeat signal. As a result, the respiratory signal corrupts the heartbeat signal by distorting the temporal morphology of the heartbeat and covering its main harmonic in time and frequency domains, respectively. To address this issue, following the band-pass filtering of the signal, a notch-filter is deployed to cancel the higher-order harmonics of respiration from the heartbeat signal. FIG. 9A describes the spectrum of the respiratory signal shown in FIG. 8A, where the waveform spikes at the respiratory frequency (f_{R}=0.151 Hz) that implies a 9.06 respirations-per-minute (RPM) rate. FIGS. 9B and 9C depict the spectrums of a heartbeat signal before and after the cancellation of the respiratory harmonics. As shown in FIG. 9B, the higher-order harmonics associated with the respiratory signal superimpose the heartbeat main harmonic. FIG. 9C illustrates the spectrum of the heartbeat signal after the cancellation of the respiratory harmonics, where the dominant peak remained within the range of 0.8-2.5 Hz holds the heartbeat frequency (f_{H}=1.492 Hz) which is equivalent to a rate of 89.52 beats-per-minute (BPM).

In some embodiments, the optical camera, depth camera, radar and a computational unit to perform signal processing and algorithmic computations to estimate heart rate and respiratory rate may be used in combination. This combination which may be packaged into one or more physical components is referred to as a camera-radar unit. In some embodiments, multiple camera-radar units may be used to monitor one or more subjects from different angles. However, two radars working in the same environment may create interference in the radar signals having a negative impact on the heart rate and respiratory rate estimation accuracy. To address this, the first camera-radar unit can communicate with a second camera-radar unit directly or indirectly through a third device to turn off the radar on the second camera-radar unit to eliminate radar signal interference.

To quantify the confidence level for the estimated respiratory and heart rates, a signal quality index is used. A signal quality index is a value representative of the reliability of the radar signal in order to extract heart rate and respiratory rate values that have acceptable accuracy. As shown in FIGS. 9A and 9C, the heart rate and respiratory rate are determined based on their respective dominant peaks. In some embodiments, the spectral signal-to-noise ratio (SNR) can be used to determine the estimation confidence metric as follow:

$${C}_{R R} = ( \frac{{\vert {X}_{R R} ( {f}_{R} ) \vert}^{2} - \overline{{\vert {X}_{R R} \vert}^{2}}}{\overline{{\vert {X}_{R R} \vert}^{2}}} ) \times 100 , \qquad ( 10 )$$

$$and$$

$${C}_{H R} = ( \frac{{\vert {X}_{H R} ( {f}_{H} ) \vert}^{2} - \overline{{\vert {X}_{H R} \vert}^{2}}}{\overline{{\vert {X}_{H R} \vert}^{2}}} ) \times 100 , \qquad ( 11 )$$

where C_{RR}, C_{HR}, X_{RR}, X_{HR}, |̅X̅_̅{̅R̅R̅}̅|̅²̅, and |̅X̅_̅{̅H̅R̅}̅|̅²̅ are defined as the respiratory rate confidence, the heart rate confidence, the spectrum of the respiratory signal, the spectrum of the heartbeat signal, the mean square of the respiratory spectrum, and the mean square of the heartbeat spectrum, respectively. In some embodiments, the degrees of periodicity

$$( \frac{{μ}_{1}}{{μ}_{2}} )$$

can be used as a signal quality index to examine the confidence of the readings. As such, the readings (also referred to as estimations) are considered valid if the signal quality index holds a value larger than a pre-defined threshold (γ). In some embodiments, the estimated values of respiratory rate and/or heart rate are recorded in the database module if the signal quality index is larger than a pre-defined threshold.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein. The appended claims should be construed broadly, to include other variants and embodiments, which may be made by those skilled in the art upon review of the technology disclosed herein.

What is claimed is:

1. A system to estimate heart rate and/or respiratory rate of a subject comprising:
a) an optical camera and a depth camera configured to receive optical and depth data of the subject in the field of view;
b) a camera processing unit communicatively coupled with the optical camera and the depth camera and a radar processing unit, wherein the camera processing unit extracts at least one anatomical landmark from the body of the subject based at least upon the optical data;
c) a radar configured to send and receive a radar signal; and
d) a radar processing unit communicatively coupled with the radar and the camera processing unit to identify a point of interest on the subject's torso and extract heart rate and respiratory rate based at least upon the movement of the point of interest.

2. The system of claim 1, further comprising a database module to record at least the estimated heart rate and/or respiratory rate of the subject.

3. The system of claim 1, wherein the radar is a frequency modulated continuous wave radar.

4. The system of claim 1, wherein the radar processing unit adjusts the point of interest on the subject's torso using a search algorithm.

5. The system of claim 4, wherein the search algorithm uses signal power or degrees of periodicity.

6. The system of claim 1, wherein the radar processing unit generates a signal quality index to quantify the confidence level for the estimated respiratory rate and/or heart rate.

7. The system of claim 2, wherein the radar processing unit generates a signal quality index to quantify the confidence level for the estimated respiratory rate and/or heart rate.

8. The system of claim 7, wherein the estimated respiratory rate and/or heart rate is recorded in the database module if the signal quality index is greater than a pre-determined threshold.

9. A computer-implemented method for estimating heart rate and/or respiratory rate of a subject, the method comprising:
a) receiving optical and depth data of the subject in the field of view;
b) extracting at least one anatomical landmark from the body of the subject based at least upon the optical data;
c) sending a radar signal;
d) receiving a radar signal and using the radar signal to identify a point of interest on the subject's torso; and
e) extracting heart rate and respiratory rate based at least upon the movement of the point of interest.

10. The method of claim 9, wherein the radar signal is generated and received by a frequency modulated continuous wave radar.

11. The method of claim 9, wherein the point of interest on the subject's torso is identified using a search algorithm.

12. The method of claim 11, wherein the search algorithm uses signal power or degrees of periodicity.

13. The method of claim 9, further comprising generating a signal quality index to quantify the confidence level for the estimated respiratory rate and/or heart rate.

14. The method of claim 13, wherein the estimated respiratory rate and/or heart rate are recorded if the signal quality index is greater than a pre-defined threshold.
